(19) United States
(12) Patent Application Publication
(10) Pub. No.: US 2025/0120913 A1
(43) Pub. Date: Apr. 17, 2025

(54) STABLE QUERCETIN NANOSUSPENSIONS

(71) Applicant: Sirona Biosciences Inc., Skillman, NJ (US)

(72) Inventors: Arvind Kumar BANSAL, Punjab (IN); Shailja DIXIT, Skillman, NJ (US); Jean Pierre LEHNER, Paris (FR); Maurice SOFEIR, Paris (FR)

(21) Appl. No.: 18/913,289

(22) Filed: Oct. 11, 2024

Related U.S. Application Data
(60) Provisional application No. 63/543,660, filed on Oct. 11, 2023.

(51) Int. Cl. A61K 9/107; A61K 31/352; A61K 47/10; A61K 47/32

(52) U.S. Cl. CPC A61K 9/1075; A61K 31/352; A61K 47/10; A61K 47/32

(57) ABSTRACT

A pharmaceutical composition of Quercetin nanosuspension comprising Quercetin dihydrate, about 0.1% w/w to 10% w/w of Poloxamer, about 0.1% w/w to 10% w/w of polyvinyl pyrrolidone, and about 0.1% w/w to 10% w/w of polyethylene glycol. A method for preparing Quercetin nanosuspension, comprising the steps of dissolving polyvinyl pyrrolidone, Poloxamer, and polyethylene glycol in purified water, adding Quercetin to the above solution, subjecting the resulting mixture to bead milling to form Quercetin nanocrystals or nanosuspension.

### CROSS-REFERENCE TO RELATED APPLICATIONS

The present application claims priority to U.S. Provisional Patent Application No. 63/543,660 filed Oct. 11, 2023, which is hereby incorporated by reference in its entirety.

### FIELD

The present invention relates to Quercetin nanosuspensions and their conversion to solid dosage forms. The invention provides an oral pharmaceutical composition comprising Quercetin (or its pharmaceutically acceptable salts) nanosuspension, which exhibits improved absorption and a fast onset of action. Without limitation to the embodiments described hereinafter, the present invention discloses a composition comprising Quercetin nanosuspension stabilized using stabilizers. Additionally, the invention provides methods for preparing the Quercetin nanosuspension and converting it into solid dosage forms through different techniques.

### BACKGROUND

Quercetin, a member of the flavonoid family, represents a class of phenolic phytochemical compounds abundantly found in natural resources. It possesses remarkable biological and pharmacological properties, including antioxidant, anti-carcinogenic, anti-inflammatory, antihypertensive, antiviral, and antibacterial activities. Notably, Quercetin stands out as the most potent flavonoid with highly efficient antioxidant effects, characterized by its ability to scavenge oxygen radicals, inhibit lipid peroxidation, chelate metal ions, and regulate cellular antioxidant responses. However, the application of Quercetin is limited due to its poor water solubility, necessitating the use of effective carriers to overcome this limitation. Quercetin exists as a solid, wherein its molecules are tightly packed and have strong intermolecular forces. The aqueous solubility of Quercetin varies from 1.5 to 12.5 mg/L depending on the pH level, which limits its oral absorption. Moreover, the octanol-water partition coefficient of Quercetin is reported to be 1.8+0.3. Additionally, Quercetin undergoes high first-pass metabolism; all these factors result in the low oral bioavailability of Quercetin.

Currently, several preparation techniques have been applied to fabricate Quercetin nanosuspensions, such as high-pressure homogenization (HPH), solvent replacement, precipitation, and wet media milling. Further, Kakran et al. studied three different fabrication methods for preparing Quercetin nanocrystals, including HPH, cavi-precipitation, and bead milling. Lai and colleagues developed Quercetin nanocrystals in the 326-474 nm range for dermal delivery using the wet milling technique. The mentioned studies mainly focused on the influence of milling time, bead size, and drug concentration. Further, several stabilizers have been described in the prior art; however, during the development of Quercetin nanosuspension, researchers of the present invention noticed a phenomenon wherein the nanosuspension gradually turned brownish. To investigate this browning effect in Quercetin nanosuspension, additional experiments were conducted. The findings suggest that the presence of some cellulosic polymers as stabilizers may be responsible for the observed browning. It is postulated that the browning phenomenon arises from the formation of quinone resonance forms, which can occur not only in Quercetin but also in other flavonoids.

Quercetin has a typical flavonoid structure and contains five hydroxyl groups. Specifically, the presence of a hydroxyl group in the para position of Ring B in Quercetin can lead to the generation of quinone forms. This conversion process is more likely to transpire in the presence of hydroxypropyl cellulose (HPC) and hydroxypropyl methylcellulose (HPMC) as stabilizers (Bansal, A., et al., 2016, Phytotherapeutic Research).

The purpose of the present invention is to develop a pharmaceutical composition with a fast onset of action and increased absorption due to the enhanced solubility of Quercetin and to develop a stable pharmaceutical composition using a simple and cost-effective manufacturing process. Further, a significant drawback of a liquid drug (nanosuspension or nanocrystal) is its limited long-term stability. Settling and Ostwald ripening are frequently recognized instability issues. Moreover, the aggregation of particles poses significant challenges during nanocrystal-based product development. The aggregation of nanocrystals can occur at various stages, including: (i) during the generation of nanocrystals; (ii) during the storage of nanosuspensions; and (iii) during the dissolution of nanocrystal-based solid dosage forms. To overcome these issues and develop a stable formulation, the nanosuspension is converted to a solid dosage form using spray drying, freeze drying, fluidized bed granulation, or granulation of diluents.

### OBJECTS AND SUMMARY OF THE INVENTION

It is an object of the present invention to formulate stable pharmaceutical compositions containing Quercetin or its pharmaceutically acceptable salts, exhibiting a fast onset of action, improved absorption, and enhanced stability.

It is an object of the present invention to develop a pharmaceutical composition with enhanced dissolution, utilizing specific excipients, including one or more stabilizers, and employing a simple and cost-effective manufacturing process.

It is an object of the present invention to develop a pharmaceutical composition with enhanced dissolution utilizing one or more stabilizers, wherein stabilizers are combinations of polymeric agents and surfactant(s).

Another object of the present invention is to create a composition comprising Quercetin nanocrystals devoid of cellulosic polymers, thereby mitigating stability concerns associated with the formulation.

Yet another object of the present invention is to design a pharmaceutical composition comprising an effective amount of Quercetin and its pharmaceutically acceptable salt in combination with a pharmaceutically acceptable carrier. The composition can be in the form of a nanosuspension, a powder that can be filled into sachets or pouches and dispersed in water prior to oral administration, or converted into tablets or capsules, among other forms.

These objectives drive the invention, which aims to provide pharmaceutical compositions of Quercetin that overcome limitations related to onset of action, absorption, stability, and permeability. By achieving these objectives, the present invention offers novel and improved formulations of Quercetin for enhanced therapeutic outcomes.

The following embodiments further describe the objects of the present invention in accordance with the best mode of practice. However, the disclosed invention is not restricted to the embodiments hereinafter described.

### DETAILED DESCRIPTION

The present invention can be more readily understood by reading the following detailed description of the invention and studying the included examples.

As used herein, drug nanocrystals are crystals with a size in the nanometer range, i.e., 10 to 1000 nm, and are composed predominantly of crystalline Quercetin stabilized with stabilizers, such as polymers and/or surfactants. Various methods exist for the preparation of nanocrystals, such as nanoprecipitation, high-pressure homogenization, and milling. These Quercetin particles have a very high drug load compared to nanoparticles consisting of a polymeric or lipidic matrix. A high drug load in nanocrystals significantly reduces the excipient load in the drug product. Nanocrystals offer apparent solubility and dissolution rate benefits by virtue of particle size reduction and increased surface area. However, an increased surface area increases the surface's free energy. To minimize the increase in surface free energy, nanocrystals tend to aggregate spontaneously. Products based on nanocrystals are mostly formulated into solid dosage forms or nanosuspensions.

“Pharmaceutical composition” and “composition,” as used herein, are equivalent terms referring to a composition of active ingredients for pharmaceutical use.

“Stable Quercetin nanosuspensions,” as used herein, means stability of the suspension against browning, which arises from the formation of quinone resonance forms.

As used herein, all numerical values relating to amounts, weights, and the like are defined as “about.” Each value is plus or minus 10%. For example, the phrase “about 10% w/w” is to be understood as “9% to 11% w/w.” Therefore, amounts within 10% of the claimed value are encompassed by the scope of the claims. As used herein, “% w/w” refers to the percent weight of the total composition. As used herein, the term “effective amount” refers to the amount necessary to treat a patient in need thereof.

The present invention shows various advantages over conventional formulations, such as:
1. The size reduction and physical stability enhancement obtained by this method impart several desirable characteristics to the products, such as longer-term physical stability of the product and chemical stability of the drug encapsulated in the nanosized dispersed phase of nanodispersion.
2. The simplicity and cost-effectiveness of the technology.
3. Improved absorption of drugs with poor aqueous solubility and thus low bioavailability.

In an embodiment of the present invention, a pharmaceutical composition of Quercetin nanosuspension comprising:
a) Quercetin dihydrate,
b) Poloxamer,
c) Polyvinyl pyrrolidone and
d) Polyethylene glycol.

In another embodiment of the present invention, a pharmaceutical composition of Quercetin nanosuspension comprising:
a) Quercetin dihydrate,
b) about 0.1% w/w to 10% w/w of Poloxamer,
c) about 0.1% w/w to 10% w/w of Polyvinyl pyrrolidone and
d) about 0.1% w/w to 10% w/w of Polyethylene glycol.

In another embodiment of the present invention, a pharmaceutical composition of Quercetin nanosuspension comprising:
a) Quercetin dihydrate,
b) about 0.1% w/w to 5% w/w of Poloxamer,
c) about 0.1% w/w to 5% w/w of Polyvinyl pyrrolidone and
d) about 0.1% w/w to 5% w/w of Polyethylene glycol.

In an embodiment of the present invention, a pharmaceutical composition of Quercetin nanosuspension comprising:
a) Quercetin dihydrate,
b) about 0.1% w/w to 2% w/w of Poloxamer,
c) about 0.1% w/w to 4% w/w of Polyvinyl pyrrolidone and
d) about 0.1% w/w to 2% w/w of Polyethylene glycol.

In another embodiment of the present invention, a pharmaceutical composition of Quercetin nanosuspension comprising:
a) Quercetin dihydrate,
b) about 0.1% w/w to 2% w/w of Poloxamer,
c) about 0.1% w/w to 2% w/w of Polyvinyl pyrrolidone and
d) about 0.1% w/w to 2% w/w of Polyethylene glycol.

In another embodiment of the present invention, a pharmaceutical composition of Quercetin nanosuspension comprises:
a) Quercetin dihydrate,
b) about 0.1% w/w to 2% w/w of Poloxamer,
c) about 0.1% w/w to 2% w/w of Polyvinyl pyrrolidone as PVPK30 and
d) about 0.1% w/w to 2% w/w of Polyethylene glycol as PEG 200.

In another embodiment of the present invention, the nanosuspension has a particle size below 400 nanometers.

In an embodiment of the present invention, the nanosuspension is devoid of cellulosic polymers.

In an embodiment of the present invention, the dose of Quercetin in the pharmaceutical composition ranges from about 100 mg to 1000 mg.

In another embodiment of the present invention, the nanosuspension is prepared using the steps of:
a) dissolving polyvinyl pyrrolidone, poloxamer, and Polyethylene glycol in purified water,
b) adding Quercetin to the above solution,
c) subjecting the resulting mixture to bead milling to form Quercetin nanocrystals or nanosuspension.

In yet another embodiment of the present invention, the Quercetin nanosuspension is converted to a solid dosage form using a method selected from the group consisting of spray drying, freeze drying, fluidized bed granulation, or granulation of diluents.

In another embodiment of the present invention, the fluidized bed granulation is performed by depositing the Quercetin nanosuspension on diluents such as microcrystalline cellulose, lactose, or mannitol.

In another embodiment of the present invention, the granulation of diluents with Quercetin nanosuspension is achieved using a rapid mixer granulator.

In another embodiment of the present invention, the final powder can be filled into sachets or pouches and dispersed in water before oral administration or converted into a tablet or capsule.

In yet another embodiment of the present invention, the composition further comprises one or more pharmaceutically acceptable excipients selected from the group consisting of fillers, binders, disintegrants, cryoprotectants, lubricants, flavouring agents, sweeteners, and glidants.

In another embodiment of the present invention, the composition is administered to a subject in need thereof, wherein it is used for its therapeutic properties selected from the group consisting of antioxidant activity, anti-carcinogenic activity, anti-inflammatory activity, antihypertensive activity, antiviral activity, antibacterial activity, and immunosuppressant activity.

In another embodiment of the present invention, the quercetin nanocrystals are characterized using one of the following techniques: energy dispersive X-Ray spectroscopy (EDX), particle size analyzer, scanning electron microscopy (SEM), transmission electron microscope (TEM), X-ray diffraction (XRD), thermogravimetric analysis (TGA), ultraviolet (UV) spectroscopy, zeta potential, dynamic light scattering (DLS), differential scanning calorimetry (DSC), atomic force microscopy (AFM), and Fourier transform infrared spectroscopy (FTIR).

In yet another embodiment of the present invention, the average particle size of the Quercetin nanocrystals ranges from about 400 nm or below, about 300 nm or below, about 200 nm or below, and about 100 nm or below. Preferably, the average particle size is about 100 nm.

In some embodiments, the quercetin composition is in solid form. The solid form of a quercetin composition can be lyophilized (freeze-dried), or alternatively, it can be spray-dried. The solid form of a quercetin composition can be provided as a powder, granules, capsules, or tablets.

The quercetin nanosuspension of the present invention is a liquid formulation wherein the drug is suspended in the form of nanoparticles. The liquid or dispersion medium is preferably an aqueous medium, such as water.

In another embodiment of the present invention, freeze-dried drug nanosuspensions help overcome the stability issue. In this way, the shelf life of the formulation with the drug in the nanoparticle range may be increased. Preferably, the freeze-dried drug nanosuspension itself has acceptable long-term stability, especially in relation to the average particle size or the particle size distribution. After storage and upon reconstitution of the freeze-dried drug nanosuspension, the average particle size, the particle size distribution, and the d50, d90, d95, or d99 are preserved or are still acceptable.

The present invention also provides a pharmaceutical composition obtained by drying the nanosuspension according to this invention. Any suitable method for drying a nanosuspension could be applied, such as, for example, but not limited to, spray-drying, solvent evaporation, hot air drying, drum drying, or vacuum drying.

Various technologies can be used to prepare nanosuspensions of the present invention like: Precipitation Methods, Antisolvent Precipitation, Reactive Precipitation; Bottom-Up Methods, Solvothermal/Hydrothermal Synthesis, Sol-Gel Method: Top-Down Methods, High-Energy Ball Milling, Bead Milling; Homogenization/High-Pressure Homogenization; Wet Media Milling using specialised milling equipment such as media mills or pearl mills; Nanoprecipitation, Emulsion/Solvent Evaporation. The preferred approach to producing a drug-nanosuspension is comminution of the drug substance in a stirred media mill by wet bead milling.

The mechanical means applied to reduce the particle size of Quercetin can be a dispersion mill. Suitable dispersion mills include, but are not limited to, a ball mill, an attritor mill, a vibratory mill, and media mills such as a sand mill or a bead mill. A bead mill is preferred due to the shorter milling time required to provide the desired reduction in particle size. The attrition time can vary widely and depends primarily upon the mechanical means and processing conditions selected.

The drug nanosuspension formulation typically comprises a stabilizer, preferably a surfactant, and a polymer. The stabilizer is adsorbed or attached to the surface of the drug nanoparticles and overcomes attractive van der Waals forces, reducing aggregation, agglomeration, or even particle fusion. This technique allows remarkable high-dose loadings of the nanosuspension.

In another embodiment of the present invention, the polymer is not a cellulosic polymer, as its interaction with quercetin leads to instability. Examples of cellulosic polymers include hydroxypropyl cellulose (HPC), an ether of cellulose, HPC ultra-low viscosity (HPC-SL), HPC-low viscosity (HPC-L), and hydroxypropyl methylcellulose (HPMC). However, it is not limited to these.

In one embodiment, the stabilizer is solid, crystalline, or amorphous at room temperature and is a combination of a polymer and surfactant. The concentration of the stabilizer in the nanosuspension may range between 1 and 200 mg/ml, between 10 and 100 mg/ml, between 10 and 75 mg/ml, between 10 and 50 mg/ml, between 20 and 50 mg/ml, or about 33.

TABLE 1
Formulas of Example 1-8

| | Ex. 1 | Ex. 2 | Ex. 3 | Ex.4 | Ex. 5 | Ex. 6 | Ex. 7 | Ex. 8 |
|---|---|---|---|---|---|---|---|---|
| Ingredients | Quantity % w/w | | | | | | | |
| Quercetin | 5.0 | 5.0 | 5.0 | 5.0 | 5.0 | 5.0 | 5.0 | 5.0 |
| Dihydrate | | | | | | | | |
| HPC ELF | — | 1.67 | 1.67 | — | — | — | — | — |
| HPMC E5 | — | — | — | — | 1.0 | — | — | — |
| HPC | — | — | — | — | — | 1.0 | — | — |
| P407 | — | 0.34 | — | 0.2 | — | — | 0.2 | 0.2 |
| P188 | — | — | — | — | 0.2 | 0.2 | 0.2 | — |
| PEG400 | — | — | 0.34 | — | — | — | — | 0.2 |
| Tween 80 | — | — | 0.83 | 0.2 | 0.2 | 0.2 | — | — |
| PVPK30 | — | — | — | 1.0 | — | — | 1.0 | 1.0 |

* # P407: Poloxamer 407; P188: Poloxamer 188; T80: Tween 80; HPC ELF: Hydroxy propyl cellulose; PVP K30: Poly vinyl pyrrolidone (Kollidon 30); HPMC: Hydroxypropyl methycellulose

TABLE 2
Results of Examples 1-8

| Parameters | Ex. 1 | Ex. 2 | Ex. 3 | Ex. 4 | Ex. 5 | Ex. 6 | Ex. 7 | Ex. 8 |
|---|---|---|---|---|---|---|---|---|
| AP | Yellow | Yellow | Light | Yellow | Yellow | Yellow | Yellow | Yellow |
| Day 0 | colour | colour | Yellowish | colour | colour | colour | colour | colour |
| | | | brown | | | | | |
| | | | colour | | | | | |
| AP | Yellow | Light | Light | Yellow | Light | Light | Yellow | Yellow |
| day 1 | colour | Yellowish | Yellowish | colour | Yellowish | Yellowish | colour | colour |
| | | brown | brown | | brown | brown | | |
| | | colour | colour | | colour | colour | | |
| AP | Yellow | Light | Light | Yellow | Light | Light | Yellow | Yellow |
| Day 2 | colour | Yellowish | Yellowish | colour | Yellowish | Yellowish | colour | colour |
| | | brown | brown | | brown | brown | | |
| | | colour | colour | | colour | colour | | |
| AP | Very light | Light | Light | Yellow | Light | Light | Yellow | Yellow |
| Day 4 | tinge of | Yellowish | Yellowish | colour | Yellowish | Yellowish | colour | colour |
| | brown | brown | brown | | brown | brown | | |
| | colour | colour | colour | | colour | colour | | |
| PS | 721.8 | 659.3 | 760 | 407. | 528.8 | 690.4 | 396. | 347. |

% drug release (minutes)

| | | | | | | | | |
|---|---|---|---|---|---|---|---|---|
| 5 | 20.85 | 59.24 | 57.77 | 56.64 | 58.32 | 61.04 | 61.91 | 79.38 |
| 10 | 22.06 | 59.71 | 58.29 | 57.14 | 57.98 | 61.90 | 62.76 | 81.91 |
| 20 | 26.19 | 60.72 | 58.60 | 57.45 | 59.52 | 62.60 | 64.35 | 83.03 |
| 40 | 36.59 | 60.59 | 60.12 | 58.94 | 59.39 | 63.17 | 65.59 | 84.64 |
| 60 | 45.45 | 61.12 | 59.11 | 57.95 | 60.26 | 63.91 | 66.10 | 90.70 |
| 120 | 36.48 | 61.48 | 60.79 | 59.59 | 59.64 | 64.18 | 67.02 | 87.31 |
| Similarity | 12.63 | 28.84 | 27.76 | 26.87 | 27.81 | 30.85 | 32.59 | 78.42 |
| factor | | | | | | | | |
| Value | | | | | | | | |

* AP.: Appearance; PS: Particle size; Disso: Dissolution

TABLE 3
Formulas of Example 8A-8F, 9 and 9A

| | Ex. 8A | Ex. 8B | Ex. 8C | Ex. 8D | Ex. 8E | Ex. 8F | Ex. 9 | Ex. 9A |
|---|---|---|---|---|---|---|---|---|
| Ingredients | Quantity % w/w | | | | | | | |
| QD | 5.0 | 5.0 | 5.0 | 5.0 | 5.0 | 5.0 | 5.0 | 5.0 |
| PVPK30 | 2.0 | 4.0 | 1.0 | 1.0 | 1.0 | — | — | — |
| P407 | 0.2 | 0.2 | 0.5 | 1.0 | — | — | 1.56 | — |
| PEG400 | 0.2 | 0.2 | 0.2 | 0.2 | 0.2 | — | — | — |
| P188 | — | — | — | — | 0.2 | 0.2 | — | 1.56 |

TABLE 4
Results of Example 8A-8F, 9 and 9A

| Parameters | Ex. 8A | Ex. 8B | Ex. 8C | Ex. 8D | Ex. 8E | Ex. 8F | Ex. 9 | Ex. 9A |
|---|---|---|---|---|---|---|---|---|
| AP | Yellow | Yellow | Yellow | Yellow | Yellow | Yellow | Yellow | Yellow |
| Day 0 | colour | colour | colour | colour | colour | colour | colour | colour |
| AP | Yellow | Yellow | Yellow | Yellow | Yellow | Yellow | Yellow | Yellow |
| day 1 | colour | colour | colour | colour | colour | colour | colour | colour |
| AP | Yellow | Yellow | Yellow | Yellow | Yellow | Yellow | Yellow | Yellow |
| Day 3 | colour | colour | colour | colour | colour | colour | colour | colour |
| AP | Yellow | Yellow | Yellow | Yellow | Yellow | Yellow | Yellow | Yellow |
| Day 4 | colour | colour | colour | colour | colour | colour | colour | colour |
| PS | 533.2 | 572.1 | 612.0 | 492.6 | 473.5 | 427.5 | 432.95 | 378.2 |
| (nm) | | | | | | | | |

% drug release (minutes)

| | | | | | | | | |
|---|---|---|---|---|---|---|---|---|
| 0 | 0 | 0 | 0 | 0 | 0 | 0 | 0 | 0 |
| 5 | 66.42 | 66.17 | 58.94 | 59.43 | 62.22 | 62.93 | 64.19 | 57.23 |
| 10 | 66.78 | 65.58 | 59.73 | 61.16 | 63.79 | 64.65 | 66.39 | 61.07 |
| 20 | 68.81 | 68.07 | 62.12 | 63.53 | 64.97 | 65.65 | 69.96 | 61.61 |
| 40 | 75.48 | 74.50 | 65.61 | 73.44 | 65.47 | 65.66 | 74.69 | 61.61 |
| 60 | 77.83 | 79.64 | 73.27 | 76.37 | 66.30 | 66.91 | 79.04 | 62.30 |
| 120 | 80.97 | 82.87 | 75.67 | 77.30 | 65.51 | 71.18 | 82.28 | 67.61 |
| F2 value | 41.95 | 41.71 | 33.58 | 36.21 | 32.65 | 34.11 | 41.81 | 30.08 |
| similarity | | | | | | | | |
| factor | | | | | | | | |

TABLE 5
Dissolution profile of the quercetin nanocrystals equivalent
to 500 mg dose of quercetin dihydrate in dissolution

| | | Ex. 8 |
|---|---|---|
| Parameters | Ex. 8 | (with 500 mg quercetin) |
| FC | QD: 5.0% w/v | QD: 5.0% w/v |
| | PVPK30: 1% w/v | PVPK30: 1% w/v |
| | P407: 0.2% w/v | P407: 0.2% w/v |
| | PEG400: 0.2% w/v | PEG400: 0.2% w/v |
| | | (n = 2) |
| Dose used in | Nanocrystals equivalent | Nanocrystals equivalent |
| dissolution | to 100 mg of the | to 500 mg of the |
| media | quercetin dihydrate | quercetin dihydrate |
| Dissolution | 900 ml of Citrate | 900 ml of Citrate |
| medium | Buffer pH 5.0 + 2% SLS | Buffer pH 5.0 + 2% SLS |
| PS (nm) | 410.2 | |
| 0 | 0 | 0 |
| 5 | 81.45 | 81.23 |
| 10 | 85.31 | 81.75 |
| 20 | 85.46 | 82.55 |
| 40 | 87.63 | 83.80 |
| 60 | 91.20 | 84.21 |
| 120 | 89.93 | 87.76 |
| F2 value | Reference dissolution | 69.96 |
| similarity | profile | |
| factor | | |

### Manufacturing Process for the Nanosuspensions

The surfactant and polymer were added to a 15-ml vial containing water. This mixture was stirred at 1500 rpm for 15 minutes at a temperature of 28° C., forming a clear solution. Quercetin was then added to the solution under continuous stirring to ensure proper dispersion. The glass vial containing the quercetin dihydrate suspension was charged with glass beads to facilitate the subsequent milling process. The aqueous suspension, containing quercetin dihydrate and glass beads, was subjected to milling at a stirring speed of 1500 rpm and a temperature of 28° C. for 7 hours. After milling, the resulting suspension was transferred to another glass vial using a pipette and stored in a refrigerator at a temperature range of 2-8° C. until further use.

Notably, the solvent used throughout the process was water, with no organic solvents involved. While glass beads were utilized for milling on a laboratory scale, zirconium beads can be employed as an alternative on a commercial scale. This process ensures the preparation of a stable nanosuspension of quercetin dihydrate, suitable for further studies and applications.

### Characterization of the Nanosuspension

The physical appearance of the nanosuspensions was monitored over 4 days for any changes in color, with results compared accordingly.

### Dissolution Studies

In the in vitro dissolution study, a USP Type 2 paddle apparatus was employed. The dosage form used consisted of liquid nanocrystals containing an equivalent of 100 mg of the active pharmaceutical ingredient. The dissolution medium was 900 mL of citrate buffer with a pH of 5.0+0.05. Sodium lauryl sulfate (SLS) was added at a concentration of 2% w/v (referred to as CBS) to enhance the dissolution process, mimicking physiological conditions. The temperature was maintained at 37° C., and the paddle rotation speed was set to 75 RPM to ensure consistency and relevance to physiological settings.

Sampling was conducted at specific time intervals: 5 minutes, 10 minutes, 20 minutes, 40 minutes, 60 minutes, and 120 minutes. At each interval, 1 mL of the dissolution medium was withdrawn for high-performance liquid chromatography (HPLC) analysis.

To prepare the samples for analysis, they were centrifuged at 15,000 RPM for 10 minutes, allowing for the separation of particulate matter. The supernatant was then filtered through a 0.22-μm membrane filter to remove any solid particles or undissolved nanocrystals. The filtered supernatant was collected and analyzed using the HPLC system.

This dissolution process enables the evaluation of the release profile and dissolution characteristics of the liquid nanocrystals, providing insights into their behavior in a simulated physiological environment.

The study examines the influence of various excipients and their concentrations on the particle size (PS), appearance, and drug release profiles of quercetin formulations. The smallest particle sizes were observed in Examples 8, 9, and 9A, indicating enhanced stability and improved dissolution rates. The dissolution data revealed that Examples 8, 8A-F, 9, and 9A, which contain specific combinations of PVP K30, Poloxamer 407, PEG 400, and Poloxamer 188, demonstrated superior drug release, with Example 8 showing the highest release rate (up to 90.70% in 60 minutes). Furthermore, the similarity factor (f2) values suggest significant differences in dissolution profiles among the formulations, with Example 8 showing the highest f2 value (78.42), indicating a dissolution profile most similar to the reference. The findings suggest that the inclusion of specific excipients in optimized concentrations significantly impacts the particle size and dissolution rate of quercetin nanosuspensions, which is critical for improving the bioavailability of poorly soluble drugs like quercetin.

Further, based on the physical appearance data and dissolution results of the formulations, it is evident that formulations utilizing cellulose polymers as stabilizers exhibited a noticeable color change to brown in the nanosuspension and a decrease in the drug release rate during dissolution studies.

This invention provides detailed information on the composition and processing of various nanosuspensions with optimal particle size and substantially improved stability across several embodiments.

What is claimed is:

1. A pharmaceutical composition of Quercetin nanosuspension comprising:
a. Quercetin dihydrate,
b. about 0.1% w/w to 10% w/w of Poloxamer,
c. about 0.1% w/w to 10% w/w of polyvinyl pyrrolidone, and
d. about 0.1% w/w to 10% w/w of polyethylene glycol.

2. The pharmaceutical composition of claim 1, wherein the nanosuspension comprises:
a. Quercetin dihydrate,
b. about 0.1% w/w to 5% w/w of Poloxamer,
c. about 0.1% w/w to 5% w/w of polyvinyl pyrrolidone, and
d. about 0.1% w/w to 5% w/w of polyethylene glycol.

3. The pharmaceutical composition of claim 1, wherein the nanosuspension comprises:
a. Quercetin dihydrate,
b. about 0.1% w/w to 2% w/w of Poloxamer,
c. about 0.1% w/w to 2% w/w of polyvinyl pyrrolidone, and
d. about 0.1% w/w to 2% w/w of polyethylene glycol.

4. The pharmaceutical composition of claim 1, wherein the nanosuspension has a particle size below 400 nanometers.

5. The pharmaceutical composition of claim 1, wherein the nanosuspension is devoid of cellulosic polymers.

6. The pharmaceutical composition of claim 1, wherein the dose of Quercetin ranges from about 100 mg to 1000 mg.

7. The pharmaceutical composition of claim 1, wherein the poloxamer is selected from P-124, P-188, P-237, P-338, and P-407.

8. The pharmaceutical composition of claim 1, wherein the polyvinyl pyrrolidone is selected from PVP K12, PVP K15, PVP K17, and PVP K30.

9. The pharmaceutical composition of claim 1, wherein the polyethylene glycol is selected from PEG 200, PEG 400, and PEG 600.

10. A method for preparing Quercetin nanosuspension, comprising the steps of:
a) dissolving polyvinyl pyrrolidone, Poloxamer, and polyethylene glycol in purified water,
b) adding Quercetin to the above solution,
c) subjecting the resulting mixture to bead milling to form Quercetin nanocrystals or nanosuspension.

11. A pharmaceutical composition of claim 10, wherein the Quercetin nanosuspension is converted to a solid dosage form using a method selected from the group consisting of spray drying, freeze drying, fluidized bed granulation, or granulation of diluents.

12. The pharmaceutical composition of claim 10, wherein the fluidized bed granulation is performed by depositing the Quercetin nanosuspension on diluents such as microcrystalline cellulose, lactose, or mannitol.

13. The pharmaceutical composition of claim 10, wherein the granulation of diluents with Quercetin nanosuspension is achieved using a rapid mixer granulator.

14. The pharmaceutical composition of claim 10, wherein the final powder is filled into sachets or pouches and dispersed in water before oral administration.

15. The pharmaceutical composition of claim 10, further comprises one or more pharmaceutically acceptable excipients selected from the group consisting of fillers, binders, disintegrants, cryoprotectants, lubricants, flavoring agents, sweeteners, and glidants.

16. A method of treating a condition, comprising administering to a subject in need thereof the pharmaceutical composition of claim 10, wherein it is used for its therapeutic properties selected from the group consisting of antioxidant activity, anti-carcinogenic activity, anti-inflammatory activity, antihypertensive activity, antiviral activity, antibacterial activity, and immunosuppressant activity.
